(19) United States
(12) Patent Application Publication
(10) Pub. No.: US 2025/0339667 A1
(43) Pub. Date: Nov. 6, 2025

(54) PULSATING STENT GRAFT WITH IMPLANTED FELXIBLE ELECTROMAGNETIC COIL OR MAGNETICALLY ACTIVATED BAND ACTUATOR TO IMPROVE CARDIAC FUNCTION AND RENAL BLOOD FLOW

(71) Applicant: PULSEGRAFT, INC., Corona Del Mar, CA (US)

(72) Inventor: Howard J. Leonhardt, Mission Viejo, CA (US)

(21) Appl. No.: 18/870,347

(22) PCT Filed: May 30, 2023
(86) PCT No.: PCT/US2023/023850

§ 371 Date: Nov. 27, 2024

Related U.S. Application Data
(60) Provisional application No. 63/348,364, filed on Jun. 2, 2022.

(51) Int. Cl. A61M 60/569; A61F 2/06; A61F 2/07; A61M 60/135; A61M 60/289; A61M 60/422; A61M 60/515; A61M 60/859; A61M 60/884

(52) U.S. Cl. CPC A61M 60/569; A61F 2/07; A61M 60/135; A61M 60/289; A61M 60/422; A61M 60/515; A61M 60/859; A61M 60/884; A61F 2002/068; A61F 2002/072; A61F 2250/0001; A61M 2205/0233; A61M 2205/0283

(57) ABSTRACT

Described is an intravascular device including a stent graft and at least one annular band. The intravascular device is configured for insertion into a blood vessel and configured to expand to contact the wall of a blood vessel after insertion therein. The annular band is configured to change in diameter in response to an applied potential difference. A method of maintaining and accelerating pulsatile blood flow includes positioning an annular band within a blood vessel, and causing the annular band to expand and contract in response to a current or potential difference.

### PRIORITY CLAIM

This application claims the benefit of the filing date of U.S. Provisional Patent Application Ser. No. 63/348,364, filed Jun. 2, 2022, the disclosure of which is hereby incorporated herein in its entirety by this reference.

### TECHNICAL FIELD

The application relates generally to electrically- or (electro)magnetically-activated pulsating stent grafts and related methods and systems. More particularly, the disclosure relates to intravascular devices that include a partial or complete annular band configured to change shape in response to an applied electrical current or intermittent magnetic force, and related methods and systems of powering and controlling them.

### BACKGROUND

Some internal circulatory assist devices have impellers (see, e.g., PCT International Patent Publication WO2019183247A1 to Leonhardt (Sep. 26, 2019) or U.S. Pat. No. 11,602,627 (Mar. 14, 2023) to Leonhardt for “Circulatory assist pump.”

The impellers of some internal circulatory assist devices however may damage red blood cells and can reduce pulsaltility of both blood flow and blood vessel wall movement. Additionally, some internal circulatory assist devices can have blood clots form within them thus blocking flow.

A pulsating vascular stent graft is disclosed in US 20220117719A1 to Leonhardt (Apr. 21, 2022). Another is disclosed in BR102012024070B1 (Aug. 3, 2021) to Agreli et al. Such pulsating vascular stent grafts help to overcome some of the deficiencies of impeller-based systems.

Some external vessel circulatory assist devices may cause damage to blood vessels, have been inconsistent in performance, and need to be placed surgically with relatively invasive procedures. Some circulatory assist devices have been known to migrate out of position, or require suture sewing, hooks, and/or barbs to be held in place. Additionally, many circulatory assist devices are too rigid for the affected artery to maintain vessel wall pulsaltility.

Many internal biomedical devices have been known to cause damage to blood cells and have a relatively high risk of leading to blood clotting and most often also eliminate arterial vessel wall or blood flow pulsaltility.

Some have tried utilizing a transmitting coil placed above a pulsating graft to power it. However, actuation by an electrical field for a device placed deep in the body requires significant power and the distance has been difficult to overcome. The higher power required to overcome resistance can cause skin irritation, significant heating, and side effects.

### DISCLOSURE

Described herein is a ventricular assist system that includes a pulsating stent graft that may, in use, avoid infection, heating of the skin, and the prior art's power and distance issues.

In certain embodiments, such a system accomplishes these goals by utilizing as a power source and a receiving coil positioned just under the skin, yet distal to a pulsating stent graft, with a corresponding transmitting coil for power positioned just above the skin, wherein the receiving coil and pulsating stent graft are connected by an electrical connection such as wires, spaced nodes, or leads.

In certain other embodiments, such a system accomplishes these goals, instead of by having wires or leads to power the pulsating stent graft, utilizing a chain, string, or succession of spaced repeaters that eventually get sufficiently close to the stent graft to power it, but do not physically contact it, which provision of power activates the movement of the pulsating stent graft via electromagnetic energy.

In one illustrative embodiment, the present disclosure provides a ventricular assist system. The ventricular assist system includes a stent graft, at least one annular band, a receiving coil, and an electrical connection. The stent graft sized for insertion into a blood vessel and configured to expand to contact the wall of a desired portion of the blood vessel after insertion therein so as to hold the stent graft in place. The at least one annular band is of an annular or semi-annular shape and encompasses at least a portion of the stent graft. The annular band(s) being configured to actuate by changing diameter in response to an applied potential difference and thus pulsing blood through the stent graft's interior. The receiving coil for implantation into a subject. The receiving coil configured to provide a sufficient potential difference to the stent graft to actuate the annular band(s). The electrical connection is between the receiving coil and the annular band(s) for powering the annular band(s).

In some embodiments, the ventricular assist system further includes a transmitting coil configured to interact with the receiving coil. In some of these embodiments, the ventricular assist system further includes a controller for the transmitting coil. In some of these embodiments, the annular band(s) include(s) an electro-activated polymer.

In some embodiments, the annular band(s) include(s) a dielectric polymer.

In some embodiments, the ventricular assist system further includes capacitors positioned in series and spaced apart along the electrical connection.

In another illustrative embodiment, the present disclosure provides a method of maintaining and supplementing pulsatile blood flow in a blood vessel of a subject. The method includes positioning an intravascular device including at least one annular band within a blood vessel, the annular band(s) including an annular or semi-annular shape. The method also includes causing the annular band(s) to expand and contract in response to a potential difference. The potential difference is provided by a receiving coil electrically connected to the annular band and configured for implantation into the subject.

In some embodiments, the intravascular device within the blood vessel comprises positioning the intravascular device within the blood vessel with a catheter. In some of embodiments, the method yet further includes delivering the potential difference on demand in pulses to cause the annular band(s) to pulsate at a chosen frequency. In some of these embodiments, the method still further includes coordinating the pulses with an electrocardiogram of the subject and/or a pacemaker associated with the patient.

In a further illustrative embodiment, the present disclosure provides a system including an electrically activated pulsating vascular stent graft, a receiving coil, and leads. The electrically activated pulsating vascular stent graft is for placement in the patient's descending thoracic aorta above the patient's kidneys. The receiving coil is for implanting under the patient's skin. The leads run from the receiving coil to the electrically activated pulsating vascular stent graft. The leads electrically connect the receiving coil with the electrically activated pulsating vascular stent graft.

In some embodiments, the system further includes a corresponding transmitting coil for positioning external to the patient's skin adjacent to the receiving coil, the transmitting coil externally powered and controlled by a processor.

In yet another illustrative embodiment, the present disclosure provides a system including an electrically activated pulsating vascular stent graft, a receiving coil, and a chain, string, or succession of spaced repeaters. The electrically activated pulsating vascular stent graft is for placement in the patient's descending thoracic aorta above the patient's kidneys. The receiving coil is for implanting under the patient's skin. The chain, string, or succession of spaced repeaters are sufficiently close to the stent graft, but not in physical contact with the stent graft, to activate the movement of the pulsating stent graft by providing electromagnetic energy.

In some embodiments, the chain, string, or succession of spaced repeaters comprises a wire with nodes spaced thereon.

In some embodiments, the chain, string, or succession of spaced repeaters comprises nodes with no wire.

In some embodiments, the system further includes an amplifier in a succession line.

### BRIEF DESCRIPTION OF THE DRAWINGS

FIG. 1 is a side elevation view of an intravascular device according to an embodiment of the disclosure.

FIG. 2 is a partial cross-sectional view of a catheter being used to insert the intravascular device of FIG. 1 into a blood vessel.

FIG. 3 is a partial cross-sectional view of the intravascular device of FIG. 1 positioned within the blood vessel in a partially actuated mode.

FIG. 4 is a partial cross-sectional view of the intravascular device of FIG. 1 positioned within the blood vessel in a fully actuated mode.

FIG. 5 depicts a non-radiative inductive coupling (NRIC) wireless power transfer (WPT) system powered by alternative electromotive force (EMF).

FIG. 6 depicts a wireless power transfer (WPT) system.

FIG. 7 depicts an alternative embodiment of the disclosure wherein series capacitors are utilized.

### MODE(S) FOR CARRYING OUT THE INVENTION

In a typical embodiment of the disclosure, a magnetically-or electrically-activated pulsating vascular stent graft is placed in the descending thoracic aorta above the patient's kidneys. A coil is also implanted under the skin (e.g., in the patient's upper leg), and electrical connections (e.g., wires or leads) run from the implanted coil up through the femoral artery and electrically connect to the stent graft to power and/or control it. Another corresponding external coil is positioned (e.g., by a belt wrapped about the patient's upper leg) on the outside of the skin immediately above and very close to the implanted coil. The external coil thus wirelessly provides power to the implanted coil, which power is transmitted to the stent graft. Therefore, the external coil can be used to power and control pulsations of the stent graft.

A programmed controller may be connected to the external coil and configured to control a current passed therethrough. In certain embodiments, the controller attempts to coordinate and synchronize the pulsations of blood from the device with the pulsations created by pumping of the heart.

In certain embodiments, the programmed controller interacts with a pacemaker implanted in the patient.

As disclosed in Palma et al. “Pulsatile stent graft: a new alternative in chronic ventricular assistance” Rev Bras Cir Cardiovasc. 2013 June; 28(2): 217-23; doi: 10.5935/1678-9741.20130031. PMID: 23939318 (see, also, BR102012024070B1 (Aug. 3, 2021) to Agreli et al.), the contents of each of which are incorporated herein by this reference, pulsatile stents composed of nickel-titanium were built and positioned to engage latex tubes. In Palma et al., different electric currents were applied to the units connected in series in order to cause structural contraction and displacement of a liquid column. Two sequence tests were conducted. The first, composed of two metallic cages, and the second composed of five cages. For the first sequence tests, a voltage of 16.3 Volts and a current of 5 Amperes was applied. In the second, a voltage of 15 volts with a current of 7 Amperes. In the first sequence, the pulsatile effect of the stent was obtained, with contraction of the tube and displacement of the water column sufficient to validate the pulsating effect of the endoprosthesis. The two structures ejected a volume of 2.6 ml per cycle, with a range of 29 mm in height of the column of water equivalent to 8% shrinkage during the pulse. In the second sequence, it reached a variation of 7.4 ml per cycle. The obtained results confirmed the stent pulsatile contractility activated by electrical current.

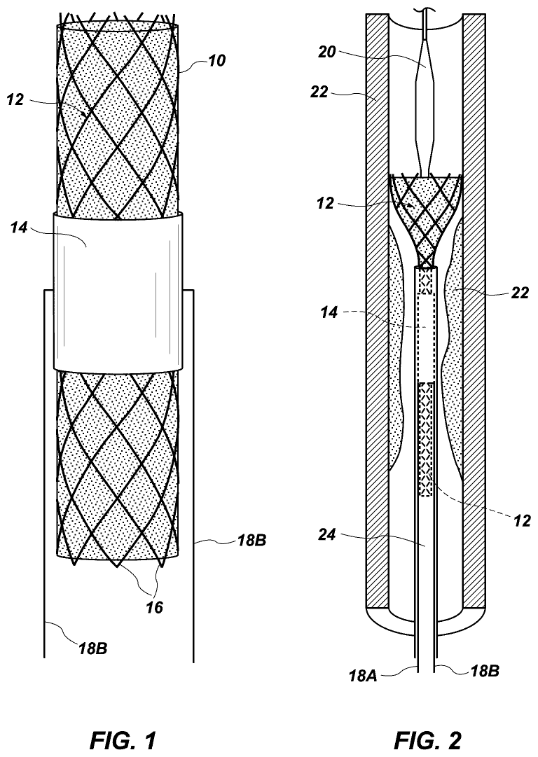

FIG. 1 shows an intravascular device 10 (also referred to as an electrically activated pulsating vascular stent graft) according to an embodiment of the disclosure. The intravascular device 10 depicted in FIG. 1 is somewhat similar to that described in the incorporated US20220117719A1 to Leonhardt, and includes a stent graft 12 and an annular band 14 or semi-annular band. In certain embodiments (not shown), the intravascular device includes one, two, three or more annular bands spaced apart along the stent graft.

The depicted annular band 14 includes an annular or semi-annular shape that radially surrounds (or partially radially surrounds) at least a portion of the stent graft 12 and is configured to be electronically (or electromagnetically) activated by application of a potential difference (e.g., an electrical current) to the annular band 14. In the depicted embodiment, the intravascular device 10 includes wires or leads 18A and 18B configured to cause the potential difference in the annular band 14.

In certain embodiments, the stent graft 12 is a GORE® stent graft with the center stent replaced with or covered by the annular band 14 and the annular band 14 includes a piezo electric band. In certain embodiments, the stent graft 12 further includes a metallic mesh covered by ePTFE (expanded polytetrafluoroethylene (PTFE) available from Gore®). See, e.g., Rosset et al. “Mechanical properties of electroactive polymer microactuators with ion-implanted electrodes” Electroactive Polymer Actuators and Devices (EAPAD) 2007, Proc. of SPIE Vol. 6524, 652410, (2007) doi: 10.1117/12.714944, the contents of which are incorporated herein by this reference.

WO 2006123317A2 to Dubois et al. (Mar. 1, 2007), the contents of which are incorporated herein by this reference, discloses a dielectric electroactive polymer comprising an elastomer layer arranged between two compliant elastomer electrodes wherein at least one of the compliant elastomer electrodes is obtained by ion implantation on the elastomer layer. The dielectric electroactive polymer may be used in an actuator, sensor, or in a power source. Also disclosed is a process for manufacturing a dielectric electroactive polymer.

In various embodiments, the leads 18A, 18B include commercially available leads, e.g., from Medtronic® (US).

In some embodiments, the annular band 14 includes an electro-activated polymer, such as a ferroelectric polymer or a dielectric polymer. Accordingly, in response to a potential difference applied to the annular band 14, the annular band 14 is configured to change shape (e.g., expand and/or contract). In the depicted embodiment, the potential difference is caused by applying a current to the annular band 14.

The depicted intravascular device 10 is sized for insertion into a blood vessel of the patient or subject (e.g., the descending thoracic aorta above the kidneys) and configured to expand to contact the wall of the blood vessel after insertion therein. The stent graft 12 may include, e.g., nitinol wires with alternating bends to form a zigzag or other shape extending circumferentially around the stent graft 12 and a graft material, such as expanded polytetrafluoroethylene (“ePTFE”), which covers the wires and may serve as an artificial blood vessel wall.

In some embodiments, the annular band 14 is comprised of a dielectric polymer or an electroactive polymer that can be actuated (e.g., to constrict) through the application of a current (e.g., a piezoelectric polymer, dielectric actuator (DEAs), electrostrictive graft elastomer, liquid crystal elastomer (LCE), ferroelectric polymer, or a combination thereof).

In various embodiments, the annular band 14 includes an outer conductive layer positioned on or proximate to the outer surface of the annular band 14 and an inner conductive layer positioned on or proximate to the inner surface of the annular band 14. In these embodiments, the dielectric polymer is positioned between the inner conductive layer and the outer conductive layer. Accordingly, when a potential difference (e.g., a current) is applied via leads 18A, 18B to the inner conductive layer and the outer conductive layer of the annular band 14 the potential difference causes the inner conductive layer and the outer conductive layer of the annular band 14 to be attracted towards one another or repulsed away from each other.

For example, application of a potential difference to the band via wires or leads 18A and 18B causes the inner conductive layer and the outer conductive layer of the annular band 14 to be attracted toward one another and the attraction of the inner conductive layer and the outer conductive layer of the annular band 14 causes the dielectric polymer positioned in between to be compressed and thinned. The compression and thinning of the dielectric polymer of the annular band 14 thus causes the diameter of the annular band to change. When the potential difference is removed or altered, the inner conductive layer and the outer conductive layer of the annular band 14 are no longer attracted to one another, and the elasticity of the dielectric polymer causes the annular band 14 to return to its original size and shape.

In another example, the application of a potential difference (e.g., appropriately selected current) via leads 18A, 18B causes the inner conductive layer and the outer conductive layer of the annular band 14 to be repelled away from one another and the repulsion of the inner conductive layer and the outer conductive layer of the annular band 14 thus causes the dielectric polymer positioned in between to be expanded and thickened. The expansion and thickening of the dielectric polymer of the annular band 14 thus causes the diameter of the annular band to decrease. When the potential difference is removed or altered, the inner conductive layer and the outer conductive layer of the annular band 14 are no longer repelled from one another, and the elasticity of the dielectric polymer causes the annular band 14 to return to its original shape.

In further embodiments, the annular band 14 includes a ferroelectric polymer, such as polyvinylidene fluoride (PVDF). Accordingly, when a potential difference (e.g., a current/voltage) is applied to the annular band 14 via leads/wires 18A, 18B, the potential difference changes the organization of the molecular dipole of the ferroelectric polymer causing a change in shape of the annular band 14. For example, the reorganization of the molecular dipoles of the ferroelectric polymer by the applied potential difference causes the annular band 14 to contract or expand. When the potential difference is no longer applied or otherwise altered, the original molecular dipole organization of the ferroelectric polymer may be at least partially restored and the annular band 14 returns to its original shape.

As shown in FIG. 2, the intravascular device 10 may be delivered to a location within a (e.g., human or mammalian) blood vessel 22 with a catheter 24 and a guide wire 20. For example, the intravascular device 10 may be delivered to a location in the blood vessel 22 with a buildup of plaque.

Prior to insertion of the catheter into a patient, the intravascular device 10 may be compressed and inserted into the catheter 24. The alternating bends in the stent wire allow the radial compression of the stent graft 12 like a spring, and the flexible polymer materials of the stent graft 12 and the annular band 14 allow sufficient deformation for positioning into the catheter 24. The guide wire 20 and leads/wires 18A, 18B may also be positioned within the catheter 24 and extend through the intravascular device 10.

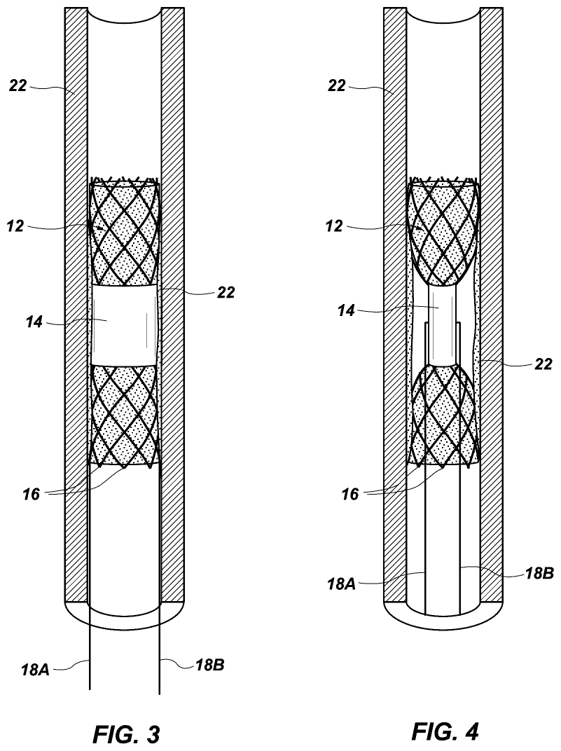

After insertion into a patient, the tip of the catheter 24 may be guided to the desired location within the blood vessel 22 with the assistance of the guide wire 20. The intravascular device 10 may then be deployed out of the tip of the catheter 24. As the stent graft 12 portion of the intravascular device 10 exits the catheter 24, the wires at the distal end may rebound like a spring and expand to cause a first end of the stent graft 12 to contact the wall of the blood vessel 22. Additionally, the stent graft 12 may expand within the annular band 14 as the annular band 14 is deployed from the catheter 24 and expands within the blood vessel 22. As the distal end of the stent graft 12 exits the catheter 24, the wires at the distal end rebound like a spring and expand to cause a second end of the stent graft 12 to contact the wall of the blood vessel 22, as shown in FIG. 3. After the intravascular device 10 has been deployed to the desired location within the blood vessel 22, the catheter 24 and the guide wire 20 (see FIG. 2) may then be withdrawn from the patient.

After the intravascular device 10 has been deployed to the desired location within the blood vessel 22, the annular band(s) 14 may be actuated to alternate between a first shape (e.g., in an expanded state), such as shown in FIG. 3, and a second shape (e.g., in a compressed state), such as shown in FIG. 4, in response to an applied potential difference to maintain and/or accelerate pulsatile blood flow. The stent graft 12 typically provides a spring radial force at the ends of the intravascular device 10 with sufficient force to secure the intravascular device 10 firmly in the blood vessel 22 without preventing vessel wall pulsaltility. Additionally, any spring force applied by the stent graft 12 under or adjacent to the annular band 14 may not be so strong as to prevent the shape change of the annular band 14 when the potential difference is applied.

In some embodiments, the at least one annular band 14 of the intravascular device 10 is powered and controlled by a device positioned outside of the patient's body via the wires/leads 18A, 18B. For example, the leads run down the blood vessel 22 and into the femoral artery. In the leg, for example, on top of the muscles of the thigh, an inductive coil 26 is implanted under the skin 28. The wires/leads 18A, 18B are in electrical connection with the implanted inductive coil. Outside of the skin, corresponding to the area very close the implanted inductive coil, is positioned a corresponding transmitter coil 30 (held in place, e.g., with a belt), which is used to power and potentially control the system. See, e.g., U.S. Pat. No. 9,642,958 to Zilbershlag et al. (May 9, 2017) for “Coplanar Wireless Energy Transfer,” the contents of which are incorporated herein by this reference. The transmitting power P_{TX} from the transmission coil is induced in the coupled receiving coil 26, the received power P_{RX} transmitted to the pulsating stent graft 12 with the use of the leads 18A, 18B.

The transmitter coil 30 is connected to a power source and potentially a controller for orchestrating the beat of the pulsating stent graft, with for example, a pacemaker of the patient.

For example, electric current may be transmitted from the conductive coil located outside of the patient's body at a location proximal to the implanted coil to generate a potential difference that may be conducted to the annular band 14 of the intravascular device 10 to activate the annular band 14 of the intravascular device 10.

For example, an external belt or other securing device worn by a patient may include a potential difference generator configured to deliver wireless electro-magnetic energy on demand in pulses to cause the annular band 14 to pulsate at a chosen frequency, which can be timed with the electrocardiogram (ECG) of the patient with delay built in. It may be understood, however, that any chosen frequency may be selected. Accordingly, the intravascular device 10 may be placed in a desired blood vessel and is used to augment blood flow providing circulatory assist support.

In some embodiments, the intravascular device 10 may be used in the aorta just above the renal arteries to help heart failure patients with excess body fluid to remove that fluid by accelerating pulsatile flow into the kidneys. In some embodiments, the intravascular device 10 may be used in legs with low blood flow to avoid limb amputation. In further embodiments, the intravascular device 10 may be used in hemodialysis patients to avoid blood clot formations in arterio-venous grafts and fistulas as well as central venous lines.

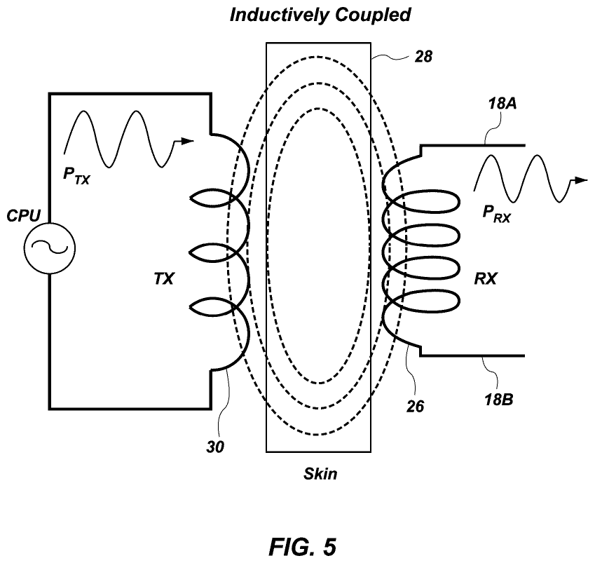

FIG. 5 (adapted from Khan, Sadeque Reza et al. “Wireless Power Transfer Techniques for Implantable Medical Devices: A Review.” Sensors (Basel, Switzerland) vol. 20,12 3487, 19 Jun. 2020, doi: 10.3390/s20123487), the contents of which are incorporated herein by this reference, depicts a transmitter (TX) coil 30 positioned adjacent to the patient's skin 28 and supplies a time varying magnetic field generated by a high-frequency voltage driver source. In some embodiments, the stent graft 12 includes a receiving (RX) coil 26. The TX coil 30 is configured to cause a magnetic field, which induces an electromotive force (EMF) in the RX coil 26 positioned within the body, which is processed using a silicon-based rectifier with the RX system. In various embodiments, the RX coil 26 is tuned to the same operating frequency as the TX coil 30 to increase the Power transfer efficiency (PTE).

In certain embodiments, the RX coil 26 is electrically connected to the at least one annular band, such as connected directly to the wires 18A and 18B that lead to the annular band 14, and power and control the constrictions of the annular band 14. In various embodiments, the TX coil 30 is positioned over the body (e.g., over or in contact with the skin 28) adjacent to the RX coil 26, such as overlapping a portion of the body within which the stent graft 12 is positioned, and in particular, overlapping a position of the RX coil 26.

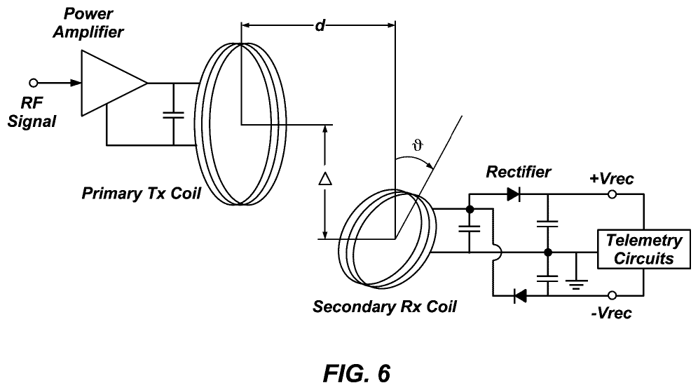

In certain embodiments, energy transfer can be obtained by using inductive or magnetic coupled systems (FIG. 6), where energy is transferred from an external primary transmitter coil (“Tx”) to a subcutaneous secondary receiver coil (“Rx”) with the aid of an alternating magnetic field. The coils are typically separated by a layer of skin and tissue in the distance of, for example, 2 to 6 cm. A typical implanted microcoil has a diameter of from 3-30 mm, whereas an external transmitter coil typically has a diameter of at least 9 cm. The Primary Tx and Secondary Rx coils should be coaxially oriented so to achieve maximum coupling results.

In certain embodiments, an energy transfer system to provide energy to the stent includes an external system including a power source and an external coil and an internal system including an internal coil adapted to receive transcutaneous energy transmitted from the external coil. Such an internal system has at least a first state wherein energy transmission from the external coil is required to provide operational power to the system. See, e.g., U.S. Pat. No. 9,308,303 to Badstilbner et al. (Apr. 12, 2016) for “Transcutaneous power transmission and communication for implanted heart assist and other devices”; U.S. Patent Publication 20140378742 to Badstilbner et al. (Dec. 25, 2014) for “Transcutaneous power transmission and communication for implanted heart assist and other devices”; U.S. Pat. No. 8,764,621 to Badstilbner et al. (Jul. 1, 2014) for “Transcutaneous power transmission and communication for implanted heart assist and other devices”; and U.S. Patent Publication 20130289334 to Badstilbner et al. (Oct. 31, 2013) for “Transcutaneous power transmission and communication for implanted heart assist and other devices.” The energy transfer system combined with the intravascular device 10 may be referred to as a ventricular assist system.

Solenoid electromagnet coils fabricated from copper or aluminum can be used for inductively coupled low-frequency passive devices operating in the range of 20-135 KHz. The coil design may include square or circular coils with multiple turns (5-10). They can be used in a form of Short Solenoid coils that are cylindrical with the diameter appreciably larger than the length. Each turn has the same radius. The coil design may also include spiral solenoid inductor coils, which are formed in flat spirals. The spiral inductors may take several forms such as square, rectangular, circular or polygonal. The coils may be imbedded in plastic substrates such as silicone or other polymers. The spacing between adjacent coil turns, the length of coils and the size Rx will depend on the size of the Tx and the distance as well as the energy required to achieve the desired constrictor effect on the graft or the stent graft. The power transfer from Tx to Rx varies depending on the coil shape and orientation.

In certain embodiments, the solenoid electromagnet coils include 8 turns of super enameled copper winding wire (“SWG copper enameled wire”) with printed spiral of strip conductor fabricated using a printed circuit board (“PCB”) technique on a single sided silicone tape with conductor thickness of 35 μm. The dielectric elastomer includes an Elastoseal film with 100-200 μm in thickness with 4 or more layers. The wireless power transfer can be achieved with a 7V-12V external battery power.

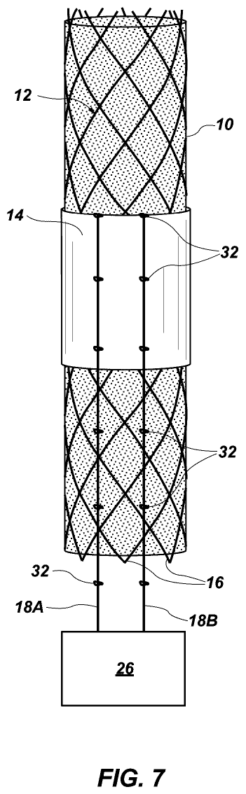

In certain embodiments, the energy transfer system includes reactive impedance elements (e.g., series capacitors) to match the impedance to the source and the load. For example, as depicted in FIG. 7, energy transfer system includes capacitors 32 positioned in series (FIG. 7) and spaced apart along the wires 18A, 18B leading from the under skin coil 26 to the intravascular device 10, and, in particular, to the annular band 14. The capacitors 32 in series in the wires 18A, 18B leading to the intravascular device 10 from the coil 16, align the current and voltage cycles transmitted to deliver all of the energy to the load the intravascular device 10, and, in particular, the annular band 14. The depicted capacitors 32 are connected in series in the circuit so as to carry the full transmission line current. In some embodiments, the capacitors 32 are arranged with a negative reactance in series with a transmission line (e.g., wires 18A or 18B). The voltage rise across each capacitor 32 is a function of circuit current and acts like a voltage regulator. Incorporating the capacitors 32 arranged in series may reduce voltage drop and “flicker” events. Once being apprised of the idea of including reactive impedance elements, one of ordinary skill in the art would be able to select and include them into the described system.

Intravascular devices 10 according to embodiments of the disclosure may be used in any application in any field where it is desired to move fluid particularly if pulsaltility of flow is desired. For example, intravascular devices according to embodiments of the disclosure may be utilized for: an aorta circulatory assist pump, a heart wrap heart assist, leg and foot circulatory flow improvement, improving blood flow into kidneys via renal artery placement, improving blood flow to the eyes, improving blood flow to the brain, improving blood vessel compliance to reduce high blood pressure, improving strength and breathing of airway tubes, reducing blood clots during hemodialysis, and improving drug delivery including cancer therapies.

When the system powers the intravascular device 10 by—instead of with wires or leads—a chain, string, or succession of spaced repeaters that are sufficiently close to the intravascular device 10 (but not in physical contact therewith) to activate the movement of the intravascular device 10 by providing electromagnetic energy, this may be via, for example, a wire with nodes spaced or via nodes with no wire; spaced so that each gap transfers energy like a synapse (e.g., there is a short gap for the energy to cross). Such a system may further utilize an amplifier in the succession line.

Embodiments of the disclosure offer improvements over other circulatory assist devices that have impellers which damage red blood cells and often reduce pulsaltility of both blood flow and blood vessel wall movement. Such devices often have blood clots form within them thus blocking flow. External vessel systems can cause damage to blood vessels, are inconsistent in performance and have to be placed via invasive surgery. Other devices migrate out of position or require suture sewing or hooks/barbs to be held in place or are too rigid to maintain vessel wall pulsaltility. Embodiments of the disclosure may avoid such problems.

In certain embodiments, the described system may include first and second power sources, e.g., batteries and control electronics for redundant uninterrupted operation of the pulsating stent graft. The control and power source module may be configured to accommodate a variety of wearable configurations for patient convenience and comfort.

In certain embodiments, the described system may be utilized with the device in a plurality of scalable power modes and/or coupling modes.

In certain embodiments, the described system may be utilized by measuring and calculating parameters to control and monitor power transfer in. The system may measure and use parameters to calculate a coupling coefficient for coils that transfer power between the external and implanted coils. The calculated coupling coefficient is used to estimate heat flux being generated in the system. Based on the level heat flux detected, the system may issue alerts to warn the subject or control actions to mitigate the effects of the heat flux.

Accordingly, embodiments of the disclosure supplement pulsatile blood flow with minimization of hemolysis and blood clot formation and without eliminating pulsatile vessel wall movement.

In the Disclosure and in the Mode(s) for Carrying Out the Invention above, the claims below, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

As used herein, the terms “configured,” and “configuration” refers to a size, a shape, a material composition, a material distribution, orientation, and arrangement of at least one feature (e.g., one or more of at least one structure, at least one material, at least one region, at least one device) facilitating use of the at least one feature in a pre-determined way.

In the application above, the claims below, and in the accompanying drawings, reference is made to particular features (including method acts) of the present disclosure. It is to be understood that the disclosure includes all possible combinations of such particular features. For example, where a particular feature is disclosed in the context of a particular embodiment, or a particular claim, that feature can also be used, to the extent possible, in combination with and/or in the context of other particular aspects and embodiments described herein.

### REFERENCES

### The Contents Of Each Of Which Are Incorporated Herein By This Reference

Khan, Sadeque Reza et al. “Wireless Power Transfer Techniques for Implantable Medical Devices: A Review.” Sensors (Basel, Switzerland) vol. 20, 12, 3487 (19 Jun. 2020), doi: 10.3390/s20123487.
Palma et al. “Pulsatile stent graft: a new alternative in chronic ventricular assistance,” Revista Brasileira de Cirurgia Cardiovascular (2013), 28 (2): 217; dx.doi.org/10.5935/1678-9741.20130031.
Rosset et al. “Mechanical properties of electroactive polymer microactuators with ion-implanted electrodes” Electroactive Polymer Actuators and Devices (EAPAD) 2007, Proc. of SPIE Vol. 6524, 652410, (2007) doi: 10.1117/12.714944.
BR102012024070B1 (Aug. 3, 2021) to Agreli et al for “Conventional Stent Graft Kit+Pulsatile Stent Graft Device for Aid to Circulatory Activity Applied to Patients with Heart Failure.”
U.S. Pat. No. 8,585,571 to Bachman (Nov. 19, 2013) for “Portable controller with integral power source for mechanical circulation support systems.”
U.S. Pat. No. 8,764,621 to Badstilbner et al. (Jul. 1, 2014) for “Transcutaneous power transmission and communication for implanted heart assist and other devices.”
U.S. Pat. No. 9,002,469 to D′Ambrosio (Apr. 7, 2015) for “Transcutaneous energy transfer system with multiple secondary coils.”
U.S. Pat. No. 9,271,825 to Arkusz et al. (Mar. 1, 2016) for “Pulsating Stent Graft.”
U.S. Pat. No. 9,308,303 to Badstilbner et al. (Apr. 12, 2016) for “Transcutaneous power transmission and communication for implanted heart assist and other devices.”
U.S. Pat. No. 9,642,958 to Zilbershlag et al. (May 9, 2017) for “Coplanar Wireless Energy Transfer.”
U.S. Pat. No. 9,855,376 to Bluvshtein et al. (Jan. 2, 2018) for “Power Scaling.”
U.S. Pat. No. 10,149,933 to Bluvshtein et al. (Dec. 11, 2018) for “Coil parameters and control.”
U.S. Pat. No. 11,602,627 (Mar. 14, 2023) to Leonhardt for “Circulatory assist pump.”
WO 2006123317A2 to Dubois et al. (Mar. 1, 2007).
U.S. Patent Publication 20130289334 to Badstilbner et al. (Oct. 31, 2013) for “Transcutaneous power transmission and communication for implanted heart assist and other devices.”
U.S. Patent Publication 20140378742 to Badstilbner et al. (Dec. 25, 2014) for “Transcutaneous power transmission and communication for implanted heart assist and other devices.”
US Patent Publication 20220117719A1 to Leonhardt (Apr. 21, 2022) for “Pulsatile vascular stent graft.”

1. A ventricular assist system comprising:
a stent graft sized for insertion into a blood vessel and configured to expand to contact a wall of a desired portion of the blood vessel after insertion therein so as to hold the stent graft in place;
at least one annular band, of an annular or semi-annular shape, encompassing at least a portion of the stent graft, the annular band(s) being configured to actuate by changing diameter in response to an applied potential difference and thus pulsing blood through the stent graft's interior;
a receiving coil for implantation into a subject, the receiving coil configured to provide a sufficient potential difference to the stent graft to actuate the annular band(s); and
an electrical connection between the receiving coil and the annular band(s) for powering the annular band(s).

2. The ventricular assist system of claim 1, further comprising a transmitting coil configured to interact with the receiving coil.

3. The ventricular assist system of claim 2, further comprising a controller for the transmitting coil.

4. The ventricular assist system of claim 1, wherein the annular band(s) include(s) an electro-activated polymer.

5. The ventricular assist system of claim 1, wherein the annular band(s) include(s) a dielectric polymer.

6. The ventricular assist system of claim 1, further comprising capacitors positioned in series and spaced apart along the electrical connection.

7. A method of maintaining and supplementing pulsatile blood flow in a blood vessel of a subject, the method comprising:
positioning an intravascular device including at least one annular band within a blood vessel, the annular band(s) including an annular or semi-annular shape; and
causing the annular band(s) to expand and contract in response to a potential difference,
wherein the potential difference is provided by a receiving coil electrically connected to the annular band and configured for implantation into the subject.

8. The method according to claim 7, wherein positioning the intravascular device within the blood vessel comprises positioning the intravascular device within the blood vessel with a catheter.

9. The method according to claim 7, further comprising delivering the potential difference on demand in pulses to cause the annular band(s) to pulsate at a chosen frequency.

10. The method according to claim 9, further comprising coordinating the pulses with an electrocardiogram of the subject and/or a pacemaker associated with a patient.

11. A system comprising:
an electrically activated pulsating vascular stent graft for placement in a patient's descending thoracic aorta above the patient's kidneys,
a receiving coil for implanting under the patient's skin, and
leads running from the receiving coil to the electrically activated pulsating vascular stent graft,
wherein the leads electrically connect the receiving coil with the electrically activated pulsating vascular stent graft.

12. The system of claim 11, further comprising:
a corresponding transmitting coil for positioning external to the patient's skin adjacent to the receiving coil, the transmitting coil externally powered and controlled by a processor.

13. A system comprising:
an electrically activated pulsating vascular stent graft for placement in a patient's descending thoracic aorta above the patient's kidneys,
a receiving coil for implanting under the patient's skin, and
a chain, string, or succession of spaced repeaters that are sufficiently close to the stent graft, but not in physical contact with the stent graft, to activate the movement of the stent graft by providing electromagnetic energy.

14. The system of claim 13, wherein the chain, string, or succession of spaced repeaters comprises a wire with nodes spaced thereon.

15. The system of claim 13, wherein the chain, string, or succession of spaced repeaters comprises nodes with no wire.

16. The system of claim 13, further comprising an amplifier in a succession line.
